(19) United States
(12) Patent Application Publication
(10) Pub. No.: US 2025/0127466 A1
(43) Pub. Date: Apr. 24, 2025

(54) RADIATION SHIELDING SYSTEM FOR MEDICAL PROCEDURE TABLE

(71) Applicant: Egg Medical, Inc., Arden Hills, MN (US)

(72) Inventors: Robert F. Wilson, Roseville, MN (US); William J. Burmaster, Plymouth, MN (US); James Montague, Elk River, MN (US); Samuel Madill, Fridley, MN (US); John P. Gainor, Mendota Heights, MN (US); Patricia Sandekian, Hudson, WI (US); David Betker, New Brighton, MN (US); Branko Djurich, Woodbury, MN (US)

(21) Appl. No.: 18/923,944

(22) Filed: Oct. 23, 2024

Related U.S. Application Data
(60) Provisional application No. 63/592,307, filed on Oct. 23, 2023.

(51) Int. Cl. A61B 6/10; A61B 6/04

(52) U.S. Cl. CPC A61B 6/107; A61B 6/0407

(57) ABSTRACT

A shielding system for reducing scatter radiation during x-ray imaging procedures may include a drawer feature to temporarily move a shield portion laterally outward from a medical procedure table. A shielding system for reducing scatter radiation during x-ray imaging procedures may include a curtain feature to temporarily move a shield portion to enable a range of positioning of an x-ray source around a medical procedure table; in some embodiments, the shield portion may include wound wire stays to flexibly and/or temporarily conform to movement of equipment near the medical procedure table. A shielding system for reducing scatter radiation during x-ray imaging procedures may include a rail extension feature to enable mounting or flexible positioning of components near a medical procedure table; in some aspects, the rail extension may include an undercut region that facilitates placement of jam boards and the like under a patient during a procedure. A shielding system for reducing scatter radiation during x-ray imaging procedures may include a telescoping hip shield feature configured to releasably couple to a rail of a medical procedure table and to extend and retract longitudinally to vary the positioning of the telescoping shield. A shielding system for reducing scatter radiation during x-ray imaging procedures may include a head protection feature configured to pivot a shield portion about two or more axes relative to a medical procedure table.

### RELATED APPLICATIONS

This application claims priority to U.S. Provisional Patent Application No. 63/592,307, filed Oct. 23, 2023, the entire contents of which are incorporated herein by reference.

### FIELD

The present disclosure describes embodiments of a radiation shielding system for protecting physicians and other health care workers during procedures involving X-ray imaging.

### BACKGROUND

Healthcare workers in hospital or clinic x-ray laboratories are often exposed to radiation, including scatter radiation (or secondary radiation) emanating from patients undergoing x-ray imaging studies and x-ray guided treatment, such as angiograms, intravascular stenting and transcatheter heart valve therapy. Shielding to absorb x-ray photons is used to protect workers from this scatter radiation. A common form of shielding is an apron worn by the user that contains a shielding material such as lead or a polymer loaded with shielding elements such as antimony, bismuth, and tin. These aprons may not cover the entire body, leaving exposed body parts subject to irradiation. Other forms of shielding (e.g., radiation blocking eyeglasses, shin guards, arm guards, skull caps, etc.) are sometimes worn to cover sensitive areas of the body not shielded by the apron, but they may not be particularly effective. Additionally, the aprons and similar worn shielding can be heavy, which may lead to fatigue, injury, and/or other orthopedic problems for the user.

A shielding system that addresses some of the above-noted concerns is described in U.S. Pat. No. 10,016,172 to Wilson, et al., relevant portions of which are hereby incorporated by reference.

During x-ray imaging procedures, the x-ray table is frequently moved to view different parts of a patient's body. In addition, the x-ray tube and detector are usually mounted on opposite sides of a C-arm gantry, where the gantry can be rotated in up to 3 planes. The movement of equipment can pose challenges to and constraints on the use of certain types of external shields. Certain procedures may involve temporarily repositioning portions of a patient and/or of imaging equipment during the procedure; in such cases, existing external shields may not provide the needed level of flexibility to easily facilitate such repositioning.

There is a need for a shielding system that protects hospital/healthcare workers from exposure to scatter radiation, while allowing them to easily move or reposition portions of the shielding system and/or the patient during such procedures.

### SUMMARY

In general, this disclosure is directed to an apparatus and method that may be useful in reducing exposure to radiation. This disclosure describes systems and/or methods for reducing the exposure of healthcare workers to scatter radiation that may arise during the performance of certain medical imaging procedures.

Certain embodiments of this disclosure describe a shielding system for reducing scatter radiation during x-ray imaging procedures including a drawer feature to temporarily move a side shield portion laterally outward from a medical procedure table. Certain embodiments of this disclosure describe a shielding system for reducing scatter radiation during x-ray imaging procedures including a curtain feature to temporarily move a shield portion to enable a range of positioning of an x-ray source around a medical procedure table; in some embodiments, the shield portion may include wound wire stays to flexibly and/or temporarily conform to movement of equipment near the medical procedure table. Certain embodiments of this disclosure describe a shielding system for reducing scatter radiation during x-ray imaging procedures including a rail extension feature to enable mounting or flexible positioning of components near a medical procedure table; in some aspects, the rail extension may include an undercut region that facilitates placement of jam boards and the like under a patient during a procedure. Certain embodiments of this disclosure describe a shielding system for reducing scatter radiation during x-ray imaging procedures including a telescoping hip shield feature configured to releasably couple to a rail of a medical procedure table and to extend and retract longitudinally to vary the positioning of the telescoping shield. Certain embodiments of this disclosure describe a shielding system for reducing scatter radiation during x-ray imaging procedures including a head protection feature configured to pivot a shield portion about two or more axes relative to a medical procedure table.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a top perspective view of a radiation shielding system with a drawer feature (in a closed position) in accordance with an embodiment of this disclosure;

FIG. 2 is a top perspective view of a radiation shielding system with a drawer feature (in an open position) in accordance with an embodiment of this disclosure;

FIG. 3 is a top perspective view of a radiation shielding system with a side shield portion having a curtain feature (in a fully extended position) in accordance with an embodiment of this disclosure;

FIG. 4 is a top perspective view of a radiation shielding system with a side shield portion having a curtain feature (in a partially extended position) in accordance with an embodiment of this disclosure;

FIG. 5 is a top perspective view of a radiation shielding system with a side shield portion having a curtain feature (in a fully retracted position) in accordance with an embodiment of this disclosure;

FIG. 6 is a top perspective view of a rail assembly for use with a radiation shielding system in accordance with an embodiment of this disclosure;

FIG. 7 is a side view of a rail assembly for use with a radiation shielding system in accordance with an embodiment of this disclosure;

FIG. 8 is a top perspective view of a rail assembly mounted to a baseplate assembly for use with a radiation shielding system in accordance with an embodiment of this disclosure;

FIG. 9 is a rear perspective view of a telescoping shield assembly (in a collapsed configuration) for use with a radiation shielding system in accordance with an embodiment of this disclosure;

FIG. 10 is a rear perspective view of a telescoping shield assembly (in an expanded configuration) for use with a radiation shielding system in accordance with an embodiment of this disclosure;

FIG. 11 is a front perspective view of a telescoping shield assembly (in an expanded configuration) mounted to a rail of a base system for use with a radiation shielding system in accordance with an embodiment of this disclosure;

FIG. 12 is a top perspective view of a slotted tube and slider assembly for supporting a curtain shield feature of a radiation shielding system in accordance with an embodiment of this disclosure;

FIG. 13 is an enlarged, partial top perspective view of the slotted tube and slider assembly of FIG. 12 for supporting a curtain shield feature of a radiation shielding system in accordance with an embodiment of this disclosure;

FIG. 14 is an enlarged, cross-sectional view of the slotted tube and slider assembly of FIG. 12 in accordance with an embodiment of this disclosure;

FIG. 15 is a top perspective view of an articulating arm support for use with a radiation shielding system in accordance with an embodiment of this disclosure;

FIG. 16 is a top view of a jam board portion an articulating arm support for use with a radiation shielding system in accordance with an embodiment of this disclosure;

FIG. 17 is a side perspective view of a ball joint for supporting an articulating arm support for use with a radiation shielding system in accordance with an embodiment of this disclosure;

FIGS. 18A-18C are side, top, and perspective views, respectively, of a ball joint for a wrist coupling of an articulating arm support for use with a radiation shielding system in accordance with an embodiment of this disclosure;

FIGS. 19A-19G are perspective views and enlarged side views showing aspects of an arm support (non-articulating) for use with a radiation shielding system in accordance with an embodiment of this disclosure;

FIGS. 20A-20D are perspective views and enlarged side views showing aspects of a releasably mountable workbench for use with a radiation shielding system in accordance with an embodiment of this disclosure;

FIGS. 21A-21J are a series of perspective views of a positionable head shielding feature for use with a radiation shielding system in accordance with an embodiment of this disclosure; and

FIGS. 22A-22D include a perspective view and detailed front and side views of a wound wire stay feature for use with a side shield portion of a radiation shielding system in accordance with an embodiment of this disclosure.

### DETAILED DESCRIPTION

The EggNest™ Radiation Protection System (developed by Egg Medical, Arden Hills, Minnesota) is a scatter radiation protection system designed to block radiation that is generated by the x-ray emitter under the cath lab table which scatters about the room as the primary beam passes through the cath lab table and the patient. Approximately 70% of this scatter radiation that reaches the staff in the room is coming from the height of the mattress top or below. To design an effective means to block scatter radiation, it is important to block scatter coming from below the table.

A feature of the EggNest™ is the ‘flex shield’ component, relatively soft shields that hang from the EggNest™ base platform system and wrap around the head of the table. In a standard cardiac catheterization laboratory (“cath lab”), there may be a single table skirt hanging near the base of the table, but rarely is there shielding further up the table and around the head of a patient. This may leave the healthcare staff working in this area unprotected from associated scatter radiation. A version of the EggNest™ contains shields disposed in this area—the shields are relatively stiff and are mounted to metal brackets that hang on metal rails around the perimeter of the EggNest™ base platform. The EggNest™ base platform is designed to have the general shape of a person lying on the table, including a rectangular carbon fiber base that contains a mattress. The carbon fiber base includes integrated carbon fiber ‘wings’ upon which the patient arms may rest. This carbon fiber material is radiolucent so that x-ray imaging can be performed throughout the body of the patient.

The EggNest™ system has been found to be effective at blocking scatter radiation to the staff in the room. During studies conducted, use of the EggNest™ system averaged about a 90% reduction in scatter radiation. When measuring this x-ray with a dosimeter, it is possible to find ‘leaks’ in the system-areas where the scatter radiation is higher than others and where improved shielding is desired. One of those areas is near the patient's arm(s). The carbon fiber wings of the base system are not shielded so that a physician can image the arm of the patient if needed (e.g., for a radial access case), which means that radiation can pass through that wing at any time during every procedure. However, arm imaging is needed relatively infrequently during a cardiac catheterization procedure, and when it is needed, it is required only for a short period of time (e.g., when the guidewire or catheter tip is passing through the elbow or shoulder). It was desired to come up with a way to provide better shielding in the area of the arm that could be simply moved out of the way in those cases where arm imaging was needed, while providing more complete protection during the majority of the cases where that imaging was not needed.

As will be described herein, an effective way to block scatter radiation has been developed to configure the shielding as close to the x-ray emitter and to the body of the patient as possible. The design of the system is such that the flexible shielding under the table is close to the edges of the patient, greatly improving the x-ray blocking properties of the system along the edge of the table. In order to provide flexibility in imaging the patient arm, a drawer is built into the base unit. This drawer has the shielding attached to it in, so that the result of the movement of this drawer is to move the shielding away from the patient, allowing a wider field of x-ray imaging.

Disclosed herein is a shielding system for reducing scatter radiation around a medical procedure table during x-ray imaging procedures, the shielding system comprising a drawer feature, the shielding system having a shield portion that is configured to extend downward along a side of the medical procedure table, the shield portion coupled to a drawer disposed along a top edge of the medical procedure table, the drawer it configured to be pulled laterally outward from the medical procedure table to temporarily reposition the shield portion during certain imaging procedures, such as when imaging a patient's arm during radial access.

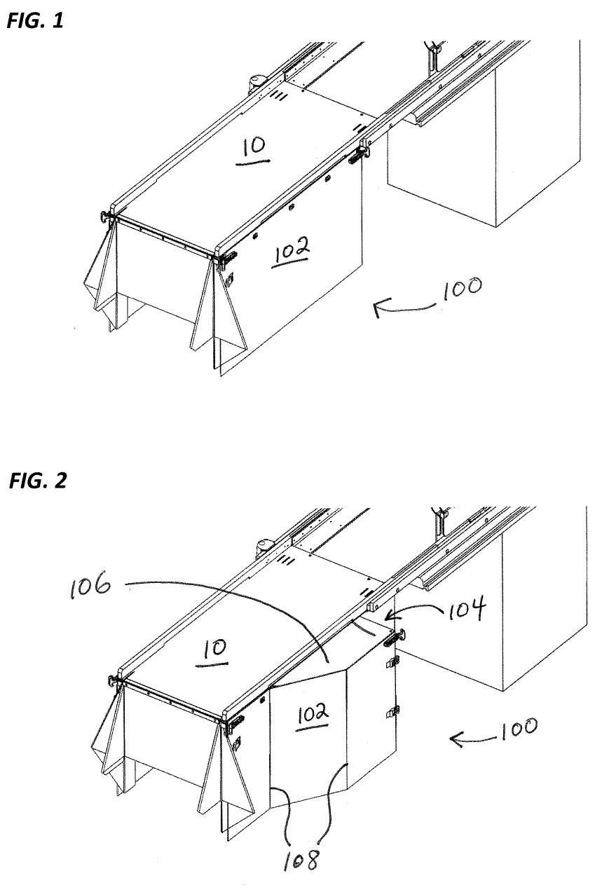

FIG. 1 is a top perspective view of a radiation shielding system 100 with a drawer feature. In the embodiment shown in FIG. 1, the drawer aspect is shown in a closed position, or a pushed in position, and the side portion 102 (also referred to as a shield portion 102, comprising a radiation shielding material) of the shielding system 100 that is shown extends downwardly from the medical procedure table 10 along a side of the medical procedure table 10 and is positioned relatively close to the side of the medical procedure table 10. This would be considered the normal position for the side portion 102 of the shielding system 100. (Although not shown in FIG. 1, a similar side portion could be disposed on an opposite side of the medical procedure table 10 and could also be configured to have a drawer or drawer feature, to be described next.)

In some situations, it may be desirable to position the side portion 102 of the shielding system 100 a distance outward from the table 10 in order to facilitate certain types of imaging. Such a situation may arise, for example, during a radial access procedure, or if complications arise during radial access. In such situations, a physician may wish to move a patient's arm outwardly from the table 10 in order to perform imaging (e.g., x-ray imaging) to troubleshoot any such complications with radial access, for example. FIG. 2 shows an example of a radiation shielding system 100 having a drawer 104 or drawer feature 104. Drawer 104 may be disposed along a top edge of table 10 and is coupled to shield portion 102. Drawer 104 is configured to be pulled outward (e.g., laterally outward from table 10, as shown in FIG. 2) to enable repositioning of the side portion 102 of the shield system 100 outward (e.g., from the normal position to an extended position) to support such temporary imaging procedures, for example. The embodiment shown in FIG. 2 shows the drawer or drawer feature 104 in an open position where a drawer panel 106 (or horizontal panel 106) is coupled to the drawer 104.

The drawer panel 106 (or horizontal panel 106) may have a generally flat surface and may be formed of a radiation shielding material according to some embodiments. Drawer panel 106 may be configured to extend horizontally outward from the medical procedure table 10 when drawer 104 is moved to the extended position. With continued reference to FIG. 2, the horizontal panel 106 and drawer 104 have been pulled laterally outward from the medical procedure table 10, and the side portion 102 of the shielding system 100 has been moved or bent at least partially outwardly with the horizontal panel 106, generally conforming to the shape of the horizontal panel 106 of the drawer feature 104. In some embodiments, the drawer panel 106 may be configured to slide laterally outward from the medical procedure table 10. In other embodiments, the drawer panel 106 may be configured to pivot about a pivot axis (not shown in FIG. 2) to enable the drawer panel 106 to extend outwardly from the medical procedure table 10.

The drawer feature 104 may thereby enable a relatively quick, temporary adjustment of the shape of the shielding system 100 to support an acute imaging need, for example, imaging of a patient's arm to troubleshoot complications with radial access, while maintaining a desirable level of scatter radiation protection for healthcare workers before, during, and after the additional imaging procedure. After the additional imaging procedure has been performed, the drawer feature 104 may be returned to its normal, inward position (e.g., closed or pushed in) to resume a normal imaging configuration. For example, the drawer 104 may be configured to be pushed laterally inward toward the medical procedure table 10 to return the side portion 102 to the normal position and to move the drawer panel 106 inward to its normal stowed position. In some embodiments, the shield portion 102 may be formed of a flexible shielding material that can bend or fold as needed during positioning of the side portion 102 between the normal position and the extended position. In some embodiments, the shield portion 102 may optionally include one or more generally vertically-oriented hinges 108 configured to facilitate bending or flexing of the shield portion 102 to generally conform to the shape of the drawer panel 106, for example.

It should be noted that the embodiments described herein may be configured for use directly with a medical procedure table, or they may involve the use of a base platform (or base unit, or base “sled”) configured to be placed on a medical procedure table in place of an existing mattress, for example. When used, such a base platform may provide support for coupling or attaching various embodiments of this disclosure thereto.

In some embodiments, it may be desirable to allow for a wider range of x-ray imaging of the patient. Typically, x-ray imaging is performed with the x-ray emitter below the table and the image intensifier above the table. The C-arm of x-ray imaging equipment can be rotated about the patient so that angles towards the left, right, head and foot can be obtained. In some cases, a lateral image of the patient is desired, when the x-ray C-arm is rotated 90 degrees with respect to the patient and an image is taken across the top of the table. In other cases, an image sequence called rotational angiography is desired where a dynamic image of the patient is taken while the C-arm rotates around the table as much as 270 degrees. During these imaging sequences, the flexible radiation shielding of certain earlier designs could sometimes get in the way of the movement of the C-arm, and the flexible radiation shielding had to be temporarily removed to perform such imaging sequences.

Disclosed herein is a shielding system for reducing scatter radiation around a medical procedure table during x-ray imaging procedures, the shielding system comprising a shield portion including a flexible shield curtain configured to extend downwardly from the side of the medical procedure table, the flexible shield curtain being slidably coupled to a rail extending along a side of the medical procedure table, the slidable coupling comprising a hollow rail having a longitudinal slot extending along a length of the hollow rail, and at least two sliders, each of the at least two sliders having a slide portion configured to slide within the longitudinal slot and a connection portion configured to extend outwardly from the longitudinal slot and engage the flexible shield curtain at a coupling point.

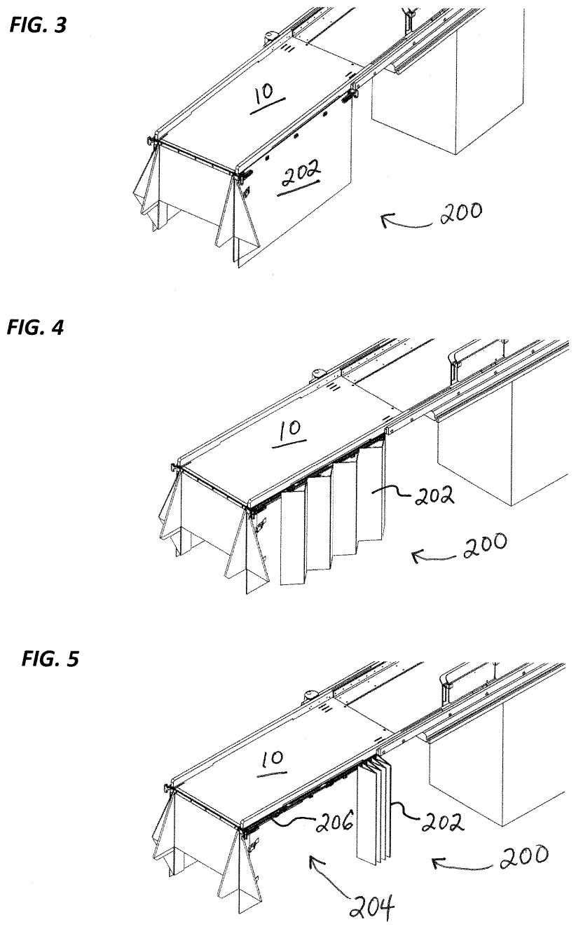

FIGS. 3-5 show an exemplary shielding system including a flexible shield curtain that can be moved (e.g., “accordioned”) along a rail 206 that extends along a side of a medical procedure table 10 (and/or a base platform in some embodiments). The flexible shield curtain may be configured to be moved to either end of rail (to the left or right as depicted in FIGS. 3-5). For example, FIG. 3 is a top perspective view of a radiation shielding system 200 with a side shield portion 202 having a flexible shield curtain, with FIG. 3 showing the side shield portion 202 in a fully extended position in accordance with an embodiment of this disclosure.

FIG. 4 is a top perspective view of a radiation shielding system 200 with a side shield portion 202 having a flexible shield curtain feature. As shown in FIG. 4, side shield portion 202 has been placed in a partially extended position by pushing the left-most portion of side shield portion 202 to the right, in accordance with an embodiment of this disclosure. The side shield portion 202 has begun to “accordion” or fold upon itself (similar to a curtain) as it begins to move toward the right in FIG. 4.

FIG. 5 shows the radiation shielding system 200 (having the flexible shield curtain feature) with the side shield portion 202 moved to a fully (or mostly) retracted position (e.g., moved fully (or mostly) to the right in the example of FIG. 5) such that the side shield portion 202 is folded upon itself and positioned to thereby provide a path or an opening 204 that may allow movement (e.g., rotation) of an x-ray C-arm around the medical procedure table 10, for example, in accordance with some embodiments of this disclosure. FIG. 5 also shows a rail 206 extending along a side of the medical procedure table 10 (and/or base platform, in some embodiments), the rail 206 being configured to support the side shield portion 202.

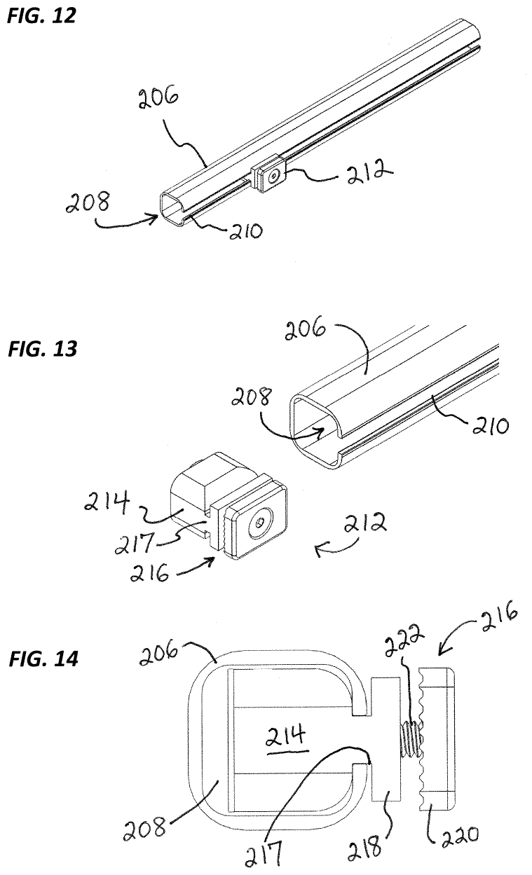

An exemplary rail 206 (such as rail 206 of FIG. 5) is shown in more detail in FIGS. 12-14. For example, FIG. 12 shows a top perspective view of a rail 206 and a slide coupling 212 (or slide coupling assembly 212) slidingly coupled to rail 206, the slide coupling 212 being configured t support a side shield portion 202 of a flexible shield curtain feature of a radiation shielding system 200 in accordance with some embodiments of this disclosure. Although FIG. 12 only shows a single slide coupling 212, in many embodiments, two or more slide couplings 212 would typically be employed to support the side shield portion 202 and to enable the folding curtain or accordion feature. As shown in the example of FIG. 12, rail 206 may be an elongate, hollow rail forming a channel 208 in an inner portion of rail 206. Rail 206 may further have a longitudinal slot 210 or opening 210 extending along a length of the rail 206 as shown. Slide coupling 212 (or slider 212), also shown in FIG. 12, is configured to slide along the longitudinal slot 210. In some embodiments, a first portion of slide coupling 212 is configured to slide within channel 208 along a length of rail 206, while a second portion of slide coupling 212 is configured to move along a length of rail 206 while disposed adjacent to an outer portion of rail 206.

FIG. 13 provides additional details regarding the arrangement of the rail 206 and the slide coupling 212. For example, FIG. 13 provides an enlarged, partial top perspective view of the rail 206, and an enlarged perspective view of an exemplary slide coupling 212. As shown, slide coupling 212 may include a slide portion 214 configured to slide within channel 208 of rail 206 along a length of rail 206. Slide coupling 212 also includes a connection portion 216 that extends from the slide portion 214 via a neck portion 217. Neck portion 217 is configured to be slidably received within longitudinal slot 210 of rail 206 such that connection portion 216 is disposed outside of rail 206. Connection portion 216 is configured to support a curtain shield feature of a radiation shielding system in accordance with an embodiment of this disclosure. For example, the connection portion 216 is configured to couple to the side shield portion 202 of the flexible shield curtain at one or more coupling points of the side shield portion 202.

FIG. 14 is an enlarged, cross-sectional view showing an exemplary slide portion 214 of the slide coupling 212 slidably received within channel 208 of rail 206, in accordance with an embodiment of this disclosure. Also depicted in the slide coupling 212 shown in FIG. 14 is an exemplary configuration of connection portion 216. For example, connection portion 216 may include a flange 218 and a clamp 220, and the clamp 220 may be configured to releasably couple and/or tighten to flange 218 via a fastener 222 (e.g., a threaded fastener 222). Connection portion 216 may facilitate coupling a flexible shield curtain (e.g., side shield portion 202) to the rail 206 via the use of flange 218, clamp 220, and fastener 222, or comparable components.

The flexible shield curtain feature described herein (with reference to FIGS. 3-5 and 12-14) provides a means for temporarily moving or retracting a flexible shield curtain (e.g., side shield portion 202) to provide an opening that may facilitate certain imaging procedures (e.g., procedures that involve having the x-ray C-arm rotate around the medical procedure table), and that does not require removal of the shielding from the system. For example, such a shielding system may include one or more rails (e.g., slotted tubes) such as rail 206 configured to be mounted to a medical procedure table, or in some cases, mounted to a base unit (or base platform, or base sled) that is disposed on a medical procedure table. The embodiments described herein refer to configurations involving either a medical procedure table or a base platform (or base unit, or base sled), but the disclosure is not so limited. In some embodiments, one or more rails may be attached to a periphery of the base platform (base sled) such that the rail extends along a side of the base platform (and along a side of the medical procedure table). The rail (or rails) may be bonded to, formed into, or otherwise attached to a base platform (base sled), according to some embodiments.

In various embodiments, the longitudinal slot 210 of rail 206 may be oriented in any direction (e.g., up, down, outwardly, inwardly, etc.). Likewise, rail 206 may be attached to a base platform (base sled) or to a medical procedure table, either on the top, the bottom, or the outer edge of the base unit or medical procedure table. In one embodiment, for example, the rail 206 may be attached to the underside of a base platform (e.g., disposed on top of a medical procedure table), and a longitudinal slot 210 may be oriented facing outwardly from the base platform. The longitudinal slot 210 of the rail 206 may be configured to carry a plurality of slide couplings 212 (e.g., as few as 1 or 2, to as many as a dozen or more, to distribute the weight of a side shield portion 202 coupled thereto) slidably disposed within a channel 208 of the rail 206. The flexible shield curtain thus described allows the side shield portion 202 to be deployed and used via the rail 206 disposed along a side of the base platform (or the medical procedure table), for example, to enhance the protection from scatter x-ray radiation, while allowing the side shield portion 202 to be retracted if needed in order to allow the x-ray C-arm to rotate around the patient during certain imaging procedures.

In some embodiments, the flexible shield curtain may be configured to flex about at least one generally vertical axis to facilitate being repositioned between an extended position and a folded position. The flexible shield curtain of side shield portion 202 can be retracted towards the head of the patient (e.g., such that the side shield portion 202 is nearer a head of the medical procedure table when in a folded position) or towards the foot of the patient (e.g., such that the side shield portion 202 is nearer a foot of the medical procedure table when in a folded position), depending on the requirements of the patient procedure. In some embodiments, the flexible shield curtain may include a retention mechanism disposed at one or both ends of the side shield portion 202 to releasably attach it to the system to prevent the shielding from being inadvertently retracted during normal use; such a retention mechanism may likewise also be used to temporarily hold the side shield portion 202 in a fully retracted position.

In some embodiments, one or more additional rails (similar to rail 206) may be placed along the head of the table or base platform. In some embodiments, a single rail 206 may be configured to extend along a first side of the table or base platform, continue around the head of the table or base platform, and extend along a second side of the table or base platform to provide greater shielding coverage around the medical procedure table, according to some embodiments.

In some embodiments, a shielding system for reducing scatter radiation around a medical procedure table during x-ray imaging procedures may comprise a shield portion 102 or 202 including a flexible shield configured to extend downwardly from the side of a medical procedure table, where the shield portion 102, 202 comprises one or more wire stays, or wound wire stays, the one or more wire stays being coupled to (or integrated into) the flexible shield portion 102 or 202 and configured to extend generally vertically from an upper portion of the flexible shield to a lower portion of the flexible shield, the one or more wire stays configured to flex or bend in various directions in response to imaging equipment coming into contact with the shield portion 102, 202, and to urge the flexible shield to return to a normal “flat” configuration in the absence of such contact.

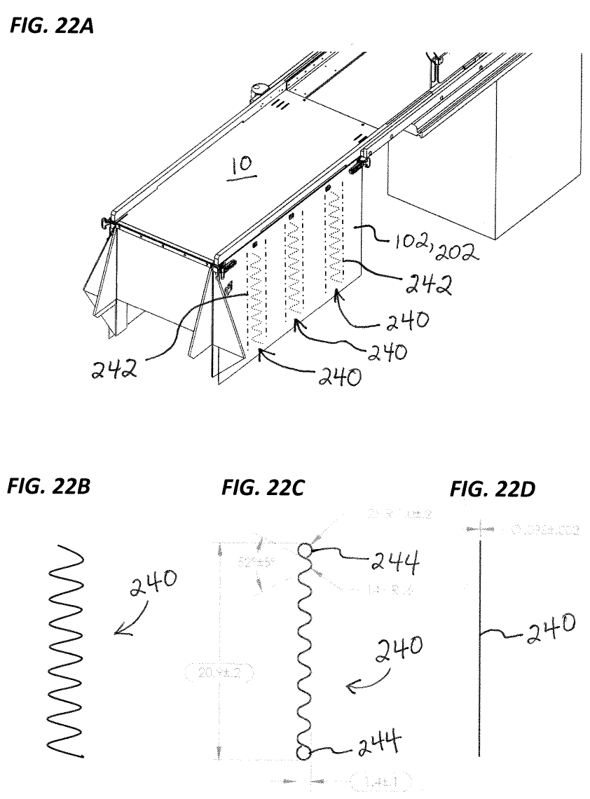

FIGS. 22A-22D illustrate the use of one or more wire stays 240 for use with a side shield portion 102, 202 of a radiation shielding system 100, 200, in accordance with some embodiments of this disclosure. For example, FIG. 22A is a top perspective view of a medical procedure table 10 showing a shield portion 102, 202 of a shielding system having a series of generally vertically-oriented pockets 242 for holding one or more wire stays 240 therewithin. FIG. 22B shows an embodiment of a wound wire stay 240 having a generally sinusoidal shape. FIGS. 22C and 22D are front and side plan views, respectively, of a wound wire stay 240, according to some embodiments of this disclosure. For example, FIG. 22C shows an embodiment of a wound wire stay 240 that is “wrapped” or curved in a loop 244 (e.g., a full loop in the particular embodiment depicted) formed at each end of wire stay 240. A looped or curved end feature 244 may help keep one or both ends of the wound wire stays 240 from damaging the fabric or material of the shield portion 102, 202 of the shielding system. The generally sinusoidal shape depicted may facilitate the wound wire stay 240 being able to bend or flex in multiple directions when the shield portion 102, 202 is acted upon by an external force (e.g., scanning equipment).

The medical procedure tables that have traditionally been used in typical cardiac catheterization laboratories (or “cath labs”) have a metallic rail that is mounted near the base of the system with a carbon fiber table top that extends in a cantilever fashion out to the head of the patient. These metallic rails generally do not extend very far along the medical procedure table towards a patient's head in order to avoid or minimize issues when imaging a patient using x-ray (e.g., to avoid such a metallic rail interfering with the path of emitted x-ray radiation). For this reason, there has typically been no shielding disposed further up the table, since there is no place to attach it to the table. Some radiation shielding designs allow for metallic rails to be mounted around the table, which may provide locations for radiation shielding to be placed. However, these metallic rails can cause imaging issues during very steep angles or during a rotational angiographic image acquisition, such as that described hereinabove. Accordingly, a need existed for a way to mount radiation shielding components to a system (such as the EggNest™ Radiation Protection System) that would allow for the full functionality of the shielding system without interfering with image acquisition.

In some embodiments, one or more radiolucent mounting rails are configured to be mounted to a carbon fiber base platform (such as the EggNest™ base platform); the radiolucent mounting rails are configured to extend the entire length of the table in some embodiments. Each mounting rail may be made of carbon fiber or a polymer that does not have a significant density such that it avoids or minimizes any x-ray image artifacts. The mounting rail may facilitate or enable the placement and use of radiation shields (e.g., extending upward from the table), and may further facilitate or enable the movement of such radiation shields (e.g., slidable movement via the mounting rail) along a side of the patient to adjust or improve shielding as needed. In some embodiments, an “undercut” or opening may be formed on a portion of the mounting rail that can enable placement of boards (e.g., jam boards) through the mounting rail and under a patient mattress; the ability to place such a jam board via the undercut in the mounting rail may provide a way to more easily support the arm of the patient, for example, or to place other devices that can be used throughout the patient imaging procedure, for example.

Disclosed herein is a mounting rail or rail assembly for use with a shielding system for a medical procedure table, the mounting rail formed of a radiolucent (e.g., polymeric) material extending along the table from the foot of a patient to the head of a patient, the mounting rail including an upwardly facing longitudinal recess extending along a length of the mounting rail to facilitate releasably supporting components mounted thereto, the mounting rail including an undercut portion configured to enable placement of one or more jam boards under a patient during an imaging procedure.

FIG. 6 is a top perspective view showing a pair of mounting rails 302 that may be used with a radiation shielding system in accordance with an embodiment of this disclosure. Each mounting rail 302 may be an elongate member formed of a radiolucent material; in some cases, the radiolucent material may be a polymeric material. The mounting rail 302 is configured to be coupled to a medical procedure table, or to a base platform 312 (see FIG. 8) according to some embodiments. The mounting rail 302 is configured to extend from a foot 308 (e.g., of a patient) to a head 310 (e.g., of a patient). The mounting rail 302 may include a means for releasably coupling one or more shielding components along a length of the elongate radiolucent member. For example, the mounting rail 302 may include an upwardly facing longitudinal recess 306, which may extend along a length of the mounting rail 302. The upwardly facing longitudinal recess 306 may be used to facilitate supporting one or more shielding components releasably mounted to the mounting rail 302 via recess 306. In some embodiments, longitudinal recess 306 may comprise an elongate channel or groove formed in an upper surface of mounting rail 302. Alternatively, the elongate radiolucent member of mounting rail 203 may present an upwardly facing shape (e.g., a cross-sectional shape) configured to be slidably received within a downward facing portion of a shielding component. In some embodiments, a coupling support may extend from a lower portion of a shielding component, the coupling support having a downward-facing channel configured to releasably and/or slidably receive the upward facing shaped portion of the mounting rail 302. In such embodiments, the channel of the coupling support may be shaped to complement the cross-sectional shape of a portion of mounting rail 302. In some embodiments, mounting rail 302 may include a parallel pair of upward facing shaped portions to provide additional mounting capabilities for shielding components. In some embodiments, the cross-sectional shape of the upward facing portion (or portions) of mounting rail 302 are rectangular. This is, however, exemplary only, and other configurations and shapes are contemplated herein.

In some embodiments, mounting rail 302 may also (or alternatively) include an undercut 304 (or undercut portion 304) formed in a lower portion of mounting rail 302. Undercut 304 may, for example, enable placement of a jam board (or other support means) through the mounting rail 302 and beneath a patient during an imaging procedure, according to some embodiments.

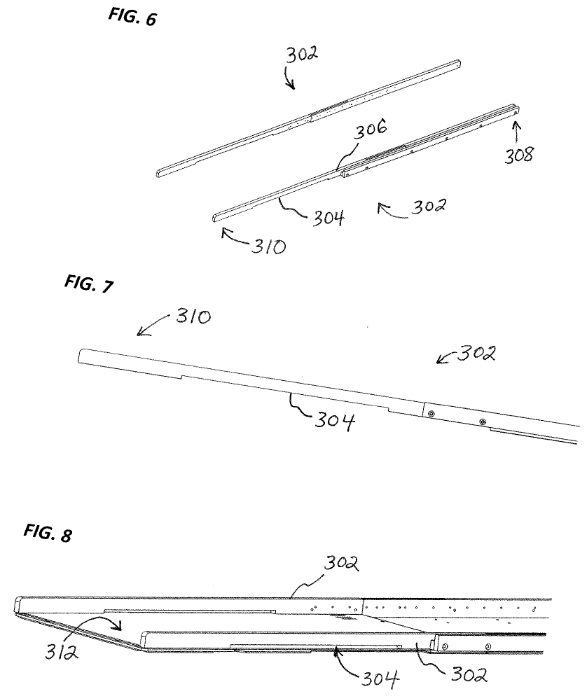

FIG. 7 is a partial side view of one of the mounting rails 302 of FIG. 6 (or mounting rail assembly 302) that may be used with a radiation shielding system in accordance with an embodiment of this disclosure. FIG. 7 shows the undercut portion 304 of the mounting rail 302, which may facilitate placement of one or more jam boards (not shown) under a patient during an imaging procedure, for example. In FIG. 7, undercut portion 304 is shown formed closer to the head portion 310 of the mounting rail 302. It should be noted that mounting rail 302 may be formed of multiple members or sections as indicated in FIGS. 6 and 7, for example.

FIG. 8 is a partial top perspective view of a pair of mounting rail assemblies 302 shown mounted to a base platform 312 or baseplate assembly 312 for use with a radiation shielding system in accordance with an embodiment of this disclosure. In the exemplary embodiment depicted, the two mounting rails 302 are mounted along each side of the base platform 312 in a longitudinally oriented configuration. Undercut 304 formed in mounting rail 302 is shown providing an opening between the mounting rail 302 and the base platform 312 through which a jam board or other temporary support means may be positioned (e.g., beneath a portion of the patient) to provide support for an arm of a patient extending outwardly, for example.

Disclosed herein is a shielding system for reducing scatter radiation around a medical procedure table during x-ray imaging procedures that includes a telescoping lateral shield portion (e.g., a telescoping hip shield) that may be configured to releasably mount to a mounting rail (such as a mounting rail 302 with one or more upwardly facing longitudinal recesses 306 as described above with respect to FIGS. 6-8). For example, a telescoping hip shield may be configured to releasably mount to a rail of a radiation shielding system (such as mounting rail 302), or to a rail of a medical procedure table. In some embodiments, a telescoping hip shield may be mounted to a mounting rail that is coupled to a base platform (such as the base platform of the EggNest™ system). The telescoping hip shield (or telescoping lateral shield portion) described herein may comprise two or more shield sections disposed adjacent to each other and configured to extend and retract relative to each other. FIGS. 9-11 provide additional details regarding an exemplary telescoping hip shield in accordance with embodiments of this disclosure.

FIG. 9 is a rear perspective view of a telescoping hip shield 400 or telescoping shield assembly 400 (shown in a collapsed or “nested” configuration) for use with a radiation shielding system in accordance with an embodiment of this disclosure. In the particular embodiment depicted in FIG. 9, telescoping shield assembly 400 includes three shield sections 402, although more or fewer shield sections 402 may be employed. FIG. 9 also shows tracks 404 and support 406 (partially hidden in FIG. 9), which are described in more detail below.

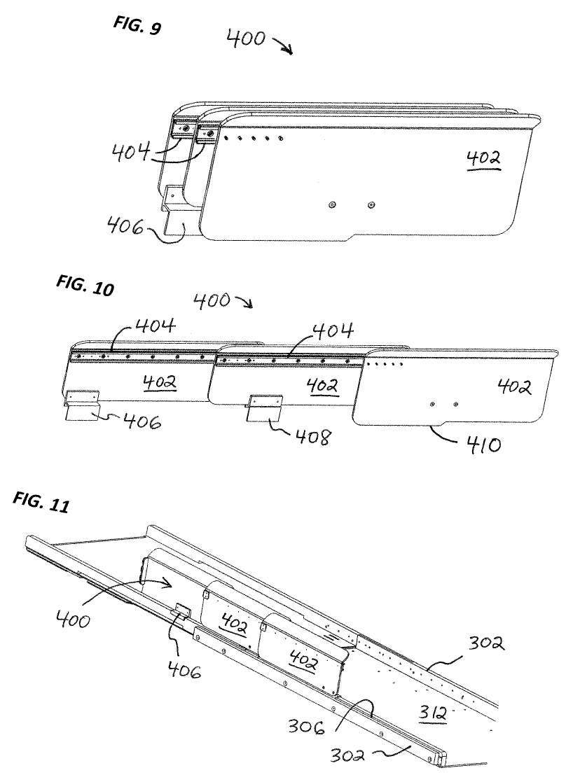

FIG. 10 is a rear perspective view of a telescoping shield assembly 400 (shown in an expanded configuration) for use with a radiation shielding system in accordance with an embodiment of this disclosure. Tracks 404 are shown mounted to two of the three shield sections 402. Tracks 404 are disposed between adjacent shield sections 402 and may provide the telescoping capability of the telescoping shield assembly 400. For example, a corresponding roller or slide member (not shown) may be disposed on a shield section 402 adjacent to the track 404 such that it is configured to roll or slide within the track 404 while expanding or collapsing the telescoping shield assembly 400, thereby maintaining alignment between adjacent shield sections 402, for example. FIG. 10 also illustrates a series of exemplary mounting mechanisms for releasably mounting the telescoping shield assembly 400 to a base platform 312, for example (scen in FIG. 11). Support 406, support 408, and tab 410 may each be configured to extend downwardly from a respective shield section 402 such that they may couple to a base platform 312 or to a medical procedure table.

FIG. 11 is a front perspective view of a telescoping shield assembly 400 (shown in an expanded configuration) releasably mounted to a mounting rail 302 that is coupled to a base platform 312 as part of a radiation shielding system in accordance with an embodiment of this disclosure. FIG. 11 provides an example of how a support 406 (possibly in conjunction with a support 408 and/or a tab 410, not seen in FIG. 11) may be releasably and/or slidably received within an upwardly facing longitudinal recess 306 of a mounting rail 302. Alternatively, a support 406 or a support 408 may form a downward-facing channel configured to releasably and/or slidably receive an upward facing portion of a mounting rail 302; in such embodiments, the channel of the support 406, 408 may be shaped to complement the cross-section shape of a portion of mounting rail 302. In some embodiments, rail 302 may include a parallel pair of upward facing portions to provide additional mounting capabilities. The embodiment of a telescoping shield assembly 400 shown in FIG. 11 may, for example, be slid down along the length of the mounting rail 302 from the expanded configuration illustrated and be nested at the foot of the table in a nested or collapsed configuration at the end of an imaging procedure (e.g., to facilitate patient transfer). Alternatively, the telescoping shield assembly 400 could be removed from the table by lifting it up from the longitudinal recess 306 of the mounting rail 302.

An articulating arm support may be used with a radiation shielding system in accordance with an embodiment of this disclosure. An articulating arm support may be useful during a radial access procedure, for example, by placing an associated jam board of the articulating arm support through the undercut of the rail. The articulation feature (e.g., provided by pivot joints and/or ball joints) may allow the patient's arm to be supported as needed by the physician for case of access during such procedures.

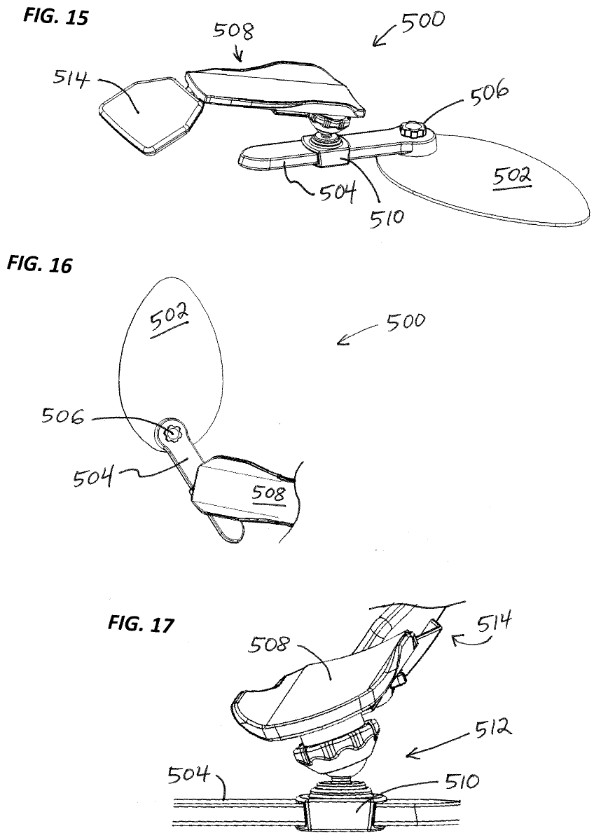

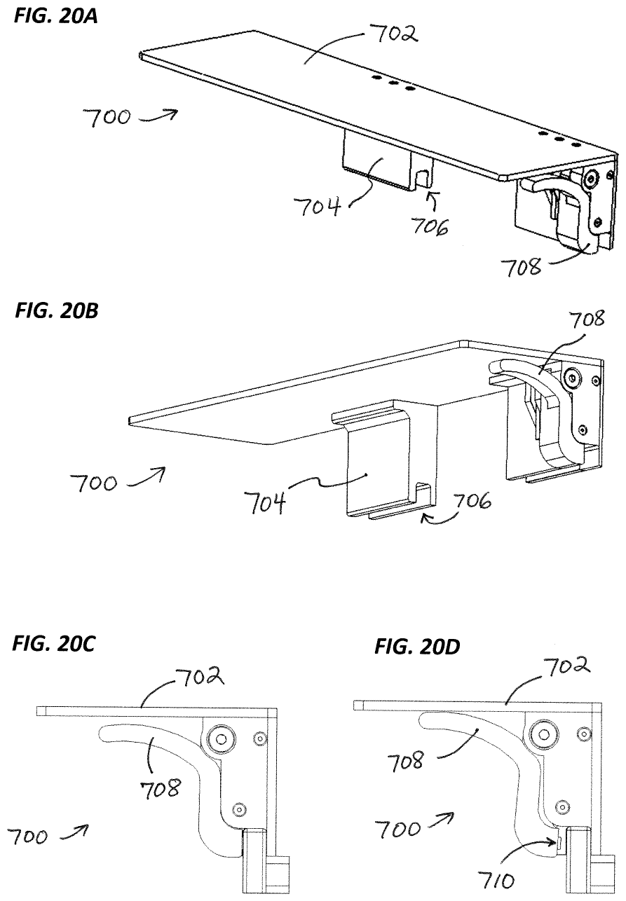

In some embodiments, it may be desirable to provide a means for positioning the arm of the patient that integrates into the radiation protection system. For radial access cases, catheter access is gained through the radial artery in the arm; proper positioning of the arm for these cases may be important for both case of performing the procedure and for the comfort of the patient. When the physician is performing catheter-based procedures through the radial access site, the catheters extend a significant distance down the table during insertion and removal. Providing a working surface that can be used in conjunction with the arm positioning system may help prevent catheters from moving out of the sterile field and may improve the workflow of the interventional procedure. An arm positioning system that can hold the arm in a natural, comfortable position that provides simple access for the physician to reach the arterial access site, while also providing a work surface upon which the physician can rest their hands and their interventional tools would provide a significant benefit to both the patient and physician. FIGS. 15-19G show a number of embodiments of an arm support, and FIGS. 20A-20D show a number of embodiments of a positionable workbench. Further aspects of these embodiments are described in more detail below.

FIG. 15 is a top perspective view of an exemplary articulating arm support 500 for use with a radiation shielding system in accordance with an embodiment of this disclosure. The articulating arm support 500 shown in FIG. 15 may include a jam board 502 (egg-shaped, flat portion), which may be configured to slide under a portion of the mattress and/or patient, for example. Arm support 500 may include an extension portion 504 rotatably coupled to the jam board 502 by a rotatable coupling 506. Arm support 500 may also include an articulating arm support portion 508 slidably coupled to the extension portion 504 via slidable coupling 510; slidable coupling 510 may be configured to be repositioned along the length of the extension portion 504 as needed to accommodate a particular patient and/or procedure.

FIG. 16 is a partial top view showing the jam board portion 502 and the extension portion 504 of an articulating arm support 500 for use with a radiation shielding system in accordance with an embodiment of this disclosure. The arm support portion 508 is supported above the extension portion 504 using a ball joint 512 (not shown in FIG. 16), shown and described below in more detail with respect to FIG. 17.

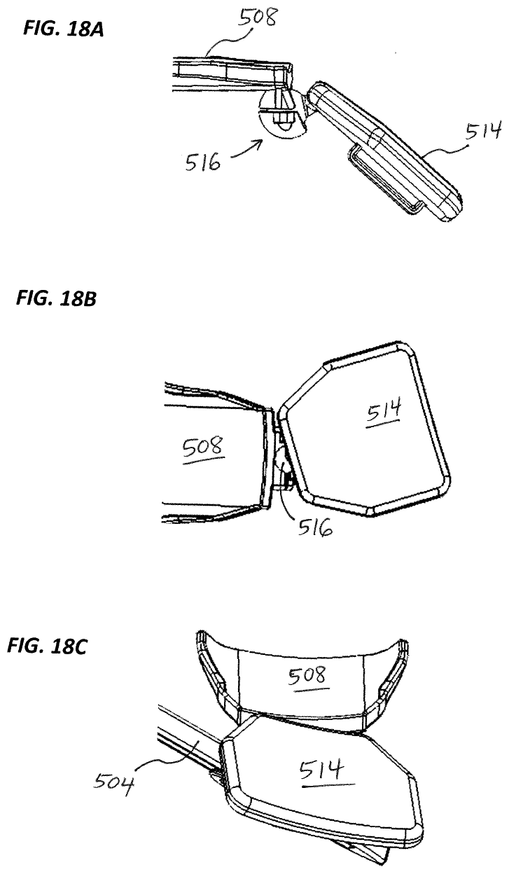

FIG. 17 is a side perspective view of a ball joint 512 for supporting a multi-axis articulating arm support 508 for use with a radiation shielding system in accordance with an embodiment of this disclosure. The ball joint 512 provides a wide range of angular adjustment options to the arm support portion 508. A wrist support portion 514 is coupled to an end of the arm support portion 508 to provide additional flexibility to position and support a patient's arm, which may be desirable during radial access procedures, for example. FIGS. 18A-18C are side, top, and perspective views, respectively, of a ball joint 516 that may be used in some embodiments for coupling a wrist support portion 514 to an articulating arm support 508 in accordance with an embodiment of this disclosure. FIGS. 18A-18C also illustrate a range of positioning options for the wrist support portion 514 relative to the arm support portion 508.

In some alternative embodiments, a releasably mountable arm support or arm rest may be used to support a patient's arm during a radial access procedure by releasably coupling the arm support to a mounting rail at or near an undercut portion of the mounting rail. The arm support can be angled outward laterally as needed by the physician to facilitate case of access. The arm support may be optionally configured to be removed from a support board portion to thereby allow the patient's arm to be placed directly on the support board in some embodiments.

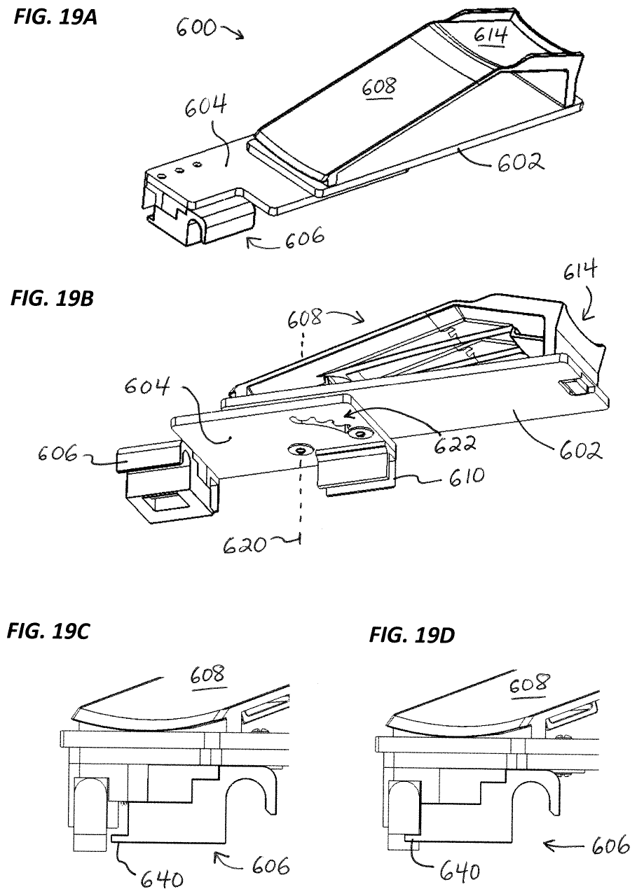

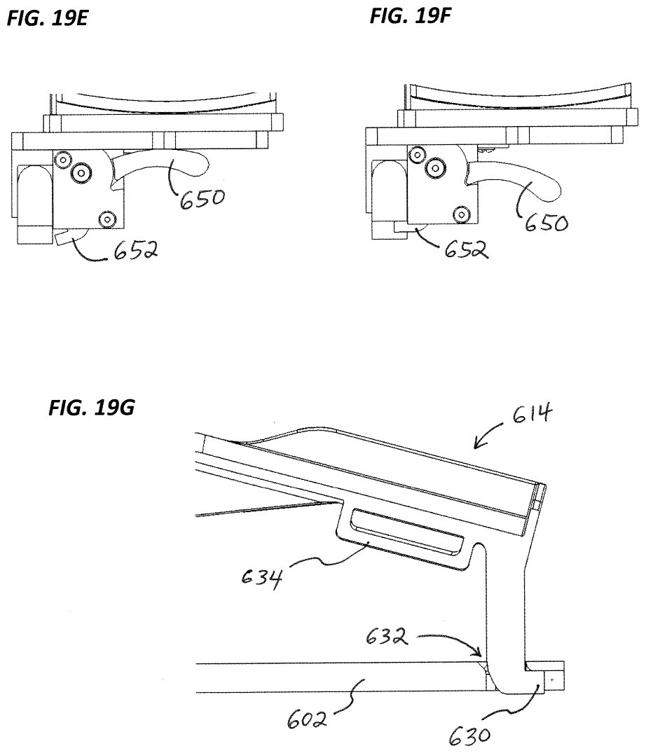

FIGS. 19A-19G include perspective views and enlarged side views showing aspects of a releasably mountable arm support 600 having an arm support portion 602 that may be configured to articulate or pivot about a single axis 620. The arm support 600 depicted may have an arm portion 608 and a wrist portion 614 coupled to the arm support portion 602, and may be configured to be used with a radiation shielding system in accordance with various embodiments of this disclosure. In some cases, the arm support portion 602 may be configured to pivot relative to a base portion 604 about a generally vertical axis 620. In FIG. 19B, for example, a series of fixed detent positions 622 (e.g., 2-5 or more discrete angular positions) may be provided for pivotable adjustment flexibility of the arm support portion 602 about axis 620 in some embodiments. In one particular example, detent positions 622 may include a range of angular positions of arm support portion 602, starting with a 0-degree position (e.g., parallel to a side of the table), and pivoting outwardly to 15, 30, 45, and 60 degree detent positions 622.

FIGS. 19C and 19D illustrate an actuatable engage-release mechanism comprising an engagement tab 640 that functions with handle 606 for releasably mounting arm support 600 to a base platform or a medical procedure table. Similarly, FIGS. 19E and 19F illustrate an actuatable engage-release mechanism comprising an engagement tab 652 that functions with lever 650 for releasably mounting arm support 600 to a base platform or a medical procedure table. FIG. 19G illustrates a top portion of the arm support having a tab 630 (e.g., an L-shaped projection) configured to fit into and engage with a corresponding slot 632 formed in the arm support portion 602 of the arm support 600 to enhance flexibility and/or support. Also shown in FIG. 19G is a loop 634 under the top portion 614 of the support 600, the loop 634 configured to guide an optional strap (not shown) that may be used to hold a patient's arm in position on the arm support 600, for example.

In some embodiments, a releasably mountable workbench may also be desirable, such that it could be positioned along a mounting rail as needed by a physician or healthcare worker or technician. A clamp (e.g., having a spring-biased mechanism) may be used in conjunction with a releasably mountable workbench to prevent unintentional movement (e.g., sliding along the rail) or dislodgement of the workbench from the rail. The worksurface height of the releasably mountable workbench may be vertically elevated to provide a shelf for catheters, wires, or leads, for example.

FIGS. 20A and 20B are perspective views, and FIGS. 20C and 20D are enlarged side views showing aspects of a releasably mountable workbench for use with a radiation shielding system in accordance with an embodiment of this disclosure. As shown, the releasably mountable workbench 700 may facilitate rapid and/or convenient placement, movement, and/or removal of the workbench 700 to and from a rail at the side of a medical procedure table. FIGS. 20A and 20B, for example, show workbench 700 having a generally horizontally oriented working surface 702 configured to extend along a side of a medical procedure table. A central support portion 704 is also shown for supporting the weight of the workbench 700 near a central portion thereof. In some configurations, a slot 706 may be formed in support portion 704 to facilitate placement and/or enhance support of the workbench when coupled to a rail of a medical procedure table. FIGS. 20C and 20D, for example, show the operation of a spring-biased lever 708 that can selectively engage and disengage the workbench 700 with the rail, for example. A protrusion 710 may be included with lever 708 to enhance the engagement with the rail (e.g., via a recess or opening in the rail, not shown) in certain embodiments. It should be noted that the spring-biased lever feature 708, if used, may be disposed on either the right or left side of the workbench 700, as desired.

Also described herein is an articulating head shield of a shielding system for reducing scatter radiation around a medical procedure table during x-ray imaging procedures, the articulating head shield comprising a plurality of shielding panels, the plurality of shielding panels including a top portion, a right side portion, and a left side portion, at least one of the right and left side portions configured to articulate or pivot about two axes.

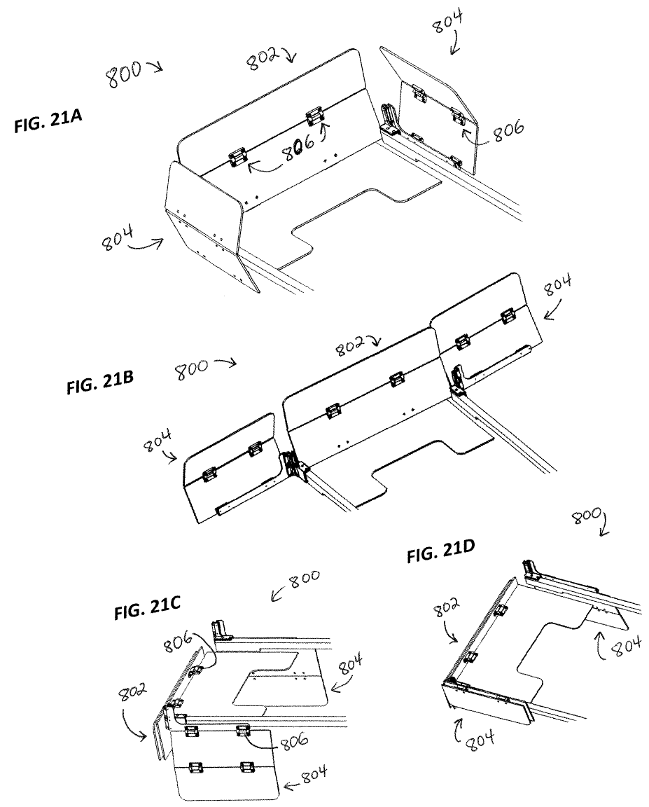

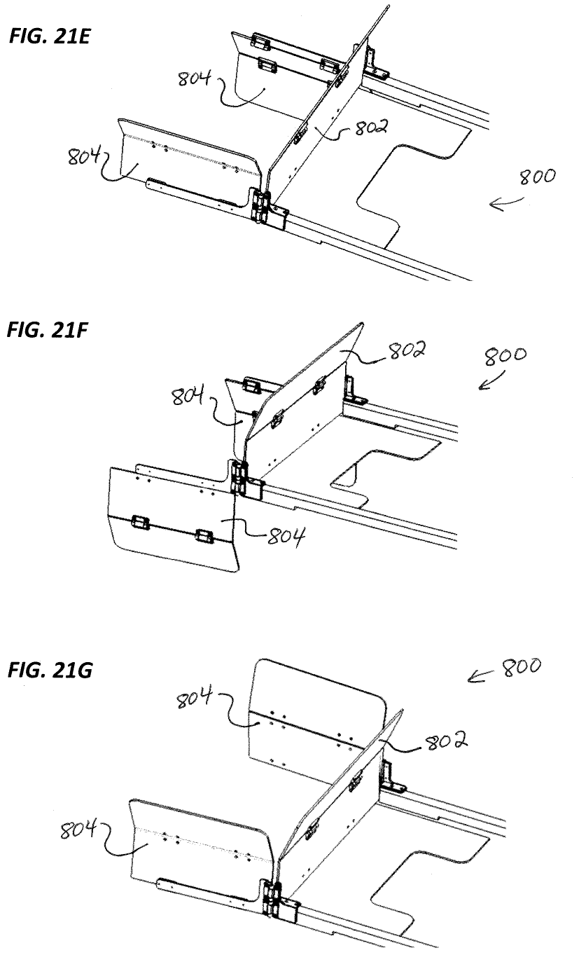

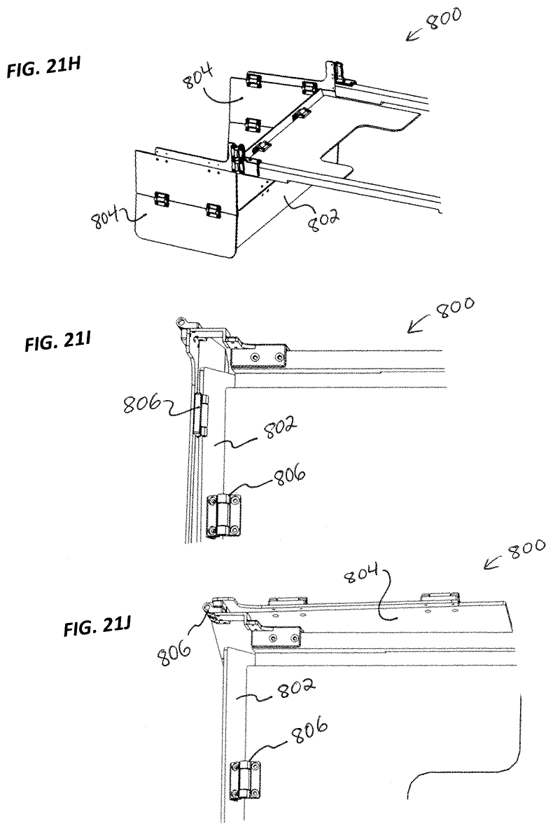

FIGS. 21A-21J are perspective views of a positionable head shielding feature, or articulating head shield 800, for use with a radiation shielding system in accordance with an embodiment of this disclosure. FIG. 21A, for example, shows a top portion 802 and right and left side portions 804 of the positionable head shield 800 in a configuration that may be typically used during a medical imaging procedure; that is, the top portion 802, left side shield portion 804, and the right side shield portion 804 of the head shield 800 are all positioned in a generally vertical or upright configuration in FIG. 21A to block or shield scatter radiation. It should be further noted that one or more of the top, right, and left side portions 802, 804 may have one or more foldable hinges 806 as depicted in FIG. 21A, which may enable making an adjustment to the height and/or shape of the particular portion of the head shield 800, as needed. FIG. 21B shows the right and left side portions 804 of the head shield 800, each having been rotated outwardly about a generally vertical axis. In some embodiments, the right and left side portions 804 of the head shield 800 may be configured to pivot outwardly about a generally vertical axis as much as 270 degrees, e.g., so that the right and/or left side portions 804 are positioned adjacent the top portion 802, according to some embodiments. FIGS. 21C and 21D show the top, right side, and left side portions 802, 804 of the head shield 800 all pivoted downwardly (e.g., about respective horizontal pivot axes) to position the portions of the head shield out of the way as needed. FIGS. 21E-21H show embodiments of the positionable head shielding feature 800 that have the various portions (top 802, right 804, and left 804 side portions) either rotated outwardly, or pivoted downwardly, or in some cases, both. FIGS. 21I and 21J are enlarged perspective views illustrating exemplary hinge and pivot arrangements to facilitate the positioning of the top, right and left side portions 802, 804 of the head shield 800 as desired.

Various examples have been described. These and other variations that would be apparent to those of ordinary skill in this field are within the scope of this disclosure.

What is claimed is:

1. A shielding system for reducing scatter radiation around a medical procedure table during imaging procedures, the shielding system comprising:
a shield portion configured to extend downwardly along a side of the medical procedure table, the shield portion comprising a radiation shielding material; and
a drawer disposed along a top edge of the medical procedure table, the drawer being coupled to the shield portion, the drawer being configured to be pulled laterally outward from the medical procedure table to reposition the shield portion from a normal position to an extended position during the imaging procedures,
wherein the shield portion is configured to flex about at least one generally vertical axis to facilitate being repositioned to the extended position.

2. The shielding system of claim 1 wherein the drawer is further configured to be pushed laterally inward toward the medical procedure table to return the shield portion to the normal position.

3. The shielding system of claim 2 wherein the drawer and the shield portion are configured to facilitate imaging a patient's arm during a radial access procedure.

4. The shielding system of claim 2 further comprising a drawer panel coupled to the drawer, the drawer panel having a generally flat surface configured to extend horizontally outward from the medical procedure table when the drawer is repositioned to the extended position.

5. The shielding system of claim 4 wherein the drawer panel is configured to slide laterally outward from the medical procedure table.

6. The shielding system of claim 4 wherein the drawer panel is configured to pivot outward from the medical procedure table.

7. The shielding system of claim 4 wherein the drawer panel is formed of a radiation shielding material.

8. The shielding system of claim 1 wherein the shield portion further comprises at least one vertically-oriented hinge.

9. A shielding system for reducing scatter radiation around a medical procedure table during x-ray imaging procedures, the shielding system comprising:
a first shield portion comprising a flexible shield curtain configured to extend downwardly from a first side of the medical procedure table;
a first rail extending along the first side of the medical procedure table, the first rail having a channel formed in the rail and a longitudinal slot extending along a length of the first rail; and
at least two slide couplings, each of the at least two slide couplings having a slide portion configured to slide within the channel of the first rail, and a connection portion configured to extend from the longitudinal slot of the first rail, the connection portion further configured to couple to the flexible shield curtain at a coupling point of the flexible shield curtain.

10. The shielding system of claim 9 wherein the flexible shield curtain is configured to flex about at least one generally vertical axis to facilitate being repositioned between an extended position and a folded position.

11. The shielding system of claim 10 wherein the flexible shield curtain in the folded position is disposed nearer a head of the medical procedure table.

12. The shielding system of claim 10 wherein the flexible shield curtain in the folded position is disposed nearer a foot of the medical procedure table.

13. The shielding system of claim 9 further comprising a second rail extending along a second side of the medical procedure table, the second rail configured to support a second shield portion configured to extend downwardly from the second side of the medical procedure table.

14. The shielding system of claim 13 further comprising an end rail extending along a head of the medical procedure table, the end rail configured to support a head shield portion extending downwardly from the head of the medical procedure table.

15. The shielding system of claim 14 wherein the first rail, the second rail, and the end rail are mounted to a base platform of the shielding system, the base platform configured to be disposed on the medical procedure table.

16. A mounting rail for use with a shielding system for a medical procedure table, the mounting rail comprising:
an elongate radiolucent member configured to be coupled to the medical procedure table, the elongate radiolucent member extending from a foot of a patient to a head of the patient, the elongate radiolucent member comprising:
a means for releasably coupling one or more shielding components along a length of the elongate radiolucent member, and
an undercut portion configured to enable placement of at least one jam board beneath the patient during an imaging procedure.

17. The mounting rail of claim 16 wherein the means for releasably coupling the one or more shielding components along the length of the elongate radiolucent member comprises an upwardly facing longitudinal recess extending along the length of the elongate radiolucent member, and wherein the elongate radiolucent member is formed of a polymeric material.

18. The mounting rail of claim 16 comprising a first elongate radiolucent member and a second elongate radiolucent member configured to be disposed generally on either side of the medical procedure table, wherein the means for releasably coupling the one or more shielding components comprises a cross-sectional shape of the elongate radiolucent member configured to be received within a channel of the one or more shielding components, the channel having a cross-sectional shape complementary to the cross-sectional shape of the elongate radiolucent member.

19. The mounting rail of claim 18, wherein the elongate radiolucent member includes a portion having a pair of parallel rectangular rails, each rectangular rail configured to be received within a rectangular channel extending from a lower portion of the one or more shielding components, the rectangular channel configured to slidingly engage one of the rectangular rails of the elongate radiolucent member.

20. A shielding system for reducing scatter radiation around a medical procedure table during x-ray imaging procedures, the shielding system comprising:
a telescoping lateral shield portion configured to releasably mount to a rail of a medical procedure table, the telescoping lateral shield portion comprising at least two shield sections disposed adjacent to each other and configured to extend and retract relative to each other.

21. The shielding system of claim 20 wherein the telescoping lateral shield portion is releasably mounted to the rail of the medical procedure table via an upwardly facing longitudinal recess extending along a length of the rail.

22. The shielding system of claim 21 wherein the telescoping lateral shield portion comprises at least one tab configured to be slidably received by the upwardly facing longitudinal recess of the rail.

23. The shielding system of claim 20 wherein the at least two shield sections of the telescoping lateral shield portion comprise a hip shield.

24. A shielding system for reducing scatter radiation around a medical procedure table during x-ray imaging procedures, the shielding system comprising:
a shield portion including a flexible shield configured to extend downwardly from the side of the medical procedure table, the flexible shield comprising one or more wire stays, the one or more wire stays being coupled to the flexible shield, the one or more wire stays configured to flex or bend in various directions in response to imaging equipment coming into contact with a portion of the flexible shield.

25. The shielding system of claim 24 wherein the one or more wire stays comprises one or more wound wire stays.

26. The shielding system of claim 25 wherein the one or more wound wire stays are configured to extend generally vertically from an upper portion of the flexible shield to a lower portion of the flexible shield.

27. The shielding system of claim 25 wherein the one or more wire stays are configured to urge the flexible shield to a normal flat position in the absence of the imaging equipment being in contact with a portion of the flexible shield.

28. An articulating head shield of a shielding system for reducing scatter radiation around a medical procedure table during x-ray imaging procedures, the articulating head shield comprising:
a plurality of shielding panels, the plurality of shielding panels including:
a top portion,
a right side portion, and
a left side portion,
wherein at least one of the right side portion and the left side portion is configured to articulate or pivot about two axes.

29. A shielding system for reducing scatter radiation around a medical procedure table during imaging procedures, the shielding system comprising:
a shield portion configured to extend along a side of the medical procedure table, the shield portion comprising a radiation shielding material, a first portion of the shield portion configured to be moved laterally outward from the medical procedure table to reposition the first portion of the shield portion from a normal position to an extended position during the imaging procedures,
wherein the shield portion is configured to flex about at least two generally vertical axes to facilitate being repositioned to the extended position.
